(19) United States
(12) Patent Application Publication
(10) Pub. No.: US 2020/0377545 A1
(43) Pub. Date: Dec. 3, 2020

(54) NOVEL ANTI-TUMOR COMPOUND AND APPLICATION THEREOF IN PREPARING ANTI-TUMOR DRUGS

(71) Applicant: HANGZHOU XINGAO BIOTECHNOLOGY CO., LTD, Hangzhou (CN)

(72) Inventors: Yingxuan TAN, Hangzhou (CN); Daofeng XIANG, Hangzhou (CN); Jianwu PEI, Hangzhou (CN); Xiangshi TAN, Hangzhou (CN)

(73) Assignee: HANGZHOU XINGAO BIOTECHNOLOGY CO., LTD

(21) Appl. No.: 16/996,891

(22) Filed: Aug. 19, 2020

Related U.S. Application Data
(63) Continuation of application No. PCT/CN2019/088455, filed on May 25, 2019.

(30) Foreign Application Priority Data
May 27, 2018 (CN) 201810527298.X

(51) Int. Cl. C07H 21/02; A61P 35/00

(52) U.S. Cl. CPC C07H 21/02; A61P 35/00

(57) ABSTRACT

The present invention belongs to the technical field of biomedicine, and discloses a novel anti-tumor compound. The anti-tumor compound is an immune activator cisplatin complex prepared from an innate immune pathway agonist and cisplatin drug molecules through a reaction. The anti-tumor drug has dual anti-tumor effects, achieves the two-pronged effects of activating innate immune pathways (generating immune T cells to fight tumors) and fighting tumors by a cisplatin chemotherapy drug, and has the significant effects of fighting tumors, increasing effect and decreasing toxicity as compared with the monofunctional cisplatin anticancer chemotherapy drug. Therefore, the novel immune activator cisplatin anti-tumor drug has an efficient clinical application prospect.

### TECHNICAL FIELD

The present invention belongs to the technical field of biomedicine, and particularly relates to a preparation method of a novel anti-tumor drug, a composition thereof, and an application thereof in preparing anti-tumor drugs.

### BACKGROUND

As one of the major diseases that seriously endanger human life and health, tumor is characterized by excessive cell proliferation and abnormal differentiation. According to the statistics from the WHO, cancer caused approximately 8.6 million deaths in 2014. In China, about 2.2 million people died of cancer. It is estimated that by 2034, the number of cancer cases worldwide may increase to 24 million annually. At the same time, cancer has caused a huge burden on the global economy. For example, the annual cost caused by cancer in 2010 was approximately $1.16 trillion. It is found through statistics that the following cancers are the chief culprits of human deaths in the world, mainly including: trachea, bronchus, lung cancer, liver cancer, gastric cancer, esophagus cancer, colorectal cancer and reproductive system cancers-prostate cancer, breast cancer and cervical cancer. Chemotherapy for cancer, referred to as chemotherapy, is a commonly used method in cancer treatment at present. Platinum drugs are chemotherapy drugs and are widely used during chemotherapy. Clinically, for different cancer patients, effective platinum drugs are selected to inhibit the growth of tumor cells or to promote tumor cells to trigger their own death mechanisms, so as to achieve the purpose of curing cancer. Therefore, a clear study of the action mechanism of platinum drugs can guide the design of novel platinum drugs with high therapeutic activity, improve the clinical treatment effect, and have great significance for the continuation of life.

Platinum anticancer drugs, as an important class of metal chemotherapy drugs, are applied to more than half of chemotherapy. Platinum anti-cancer drugs that have been approved for marketing at present include: cisplatin (cis-[PtCl₂(NH₃)₂]), carboplatin and oxaliplatin, as well as those approved in some countries, including nedaplatin (China), lobaplatin (Japan), and heptaplatin (South Korea). etc. These platinum drugs are clinically used to treat ovarian cancer, prostate cancer, testicle cancer, lung cancer, nasopharyngeal cancer, esophagus cancer, malignant lymphoma, head and neck cancer, thyroid cancer, osteogenic sarcoma and other various solid tumors. Cisplatin and similar platinum drugs thereof have significant anticancer activity in treating a series of malignant tumors, and may be combined with bleomycin, paclitaxel, 5-fluorouracil and other drugs clinically to achieve a better therapeutic effect. However, the application of platinum anticancer drugs is limited by their own toxic and side effects and the inherent and acquired drug resistance of tumor cells. How to find platinum drugs with higher efficiency and lower toxicity in the clinical treatment process becomes an urgent problem that people need to solve. Therefore, a platinum complex is prepared based on monofunctional platinum complexes, to make same have composite anti-tumor functions and can increase effect and decrease toxicity. The research of the innovative anticancer compound has become an international hot topic.

Cyclic dinucleotide synthase (cGAS) is an important cytoplasmic DNA receptor in the innate immune pathway. As a secondary messenger molecule, cGAMP induces the production of interferon IFN-β and other cytokines through the STING protein pathway on the endoplasmic reticulum membrane, regulates the expression of downstream protein, induces cell growth arrest and apoptosis, and produces an antiviral effect. The STING pathway can regulate the innate immune recognition of immunogenic tumors and promote the anti-tumor effect of interferon. IFN-γ plays an anti-tumor effect in vivo through TRAIL (tumor necrosisfactor-related apoptosis-inducing ligand), and promotes the apoptosis of tumor cells. cGAMP is a key irritant of innate immune response and an endogenous activator of the STING. Therefore, cGAMP has an immune anti-tumor effect.

### SUMMARY

The purpose of the present invention is to provide a composition of a novel anti-tumor compound, a preparation method thereof, and an application thereof in fighting tumors and preparing anti-tumor drugs. The composite anti-tumor drug is prepared from platinum complexes with a monofunctional anticancer effect and an activator (/agonist) of the STING innate immune pathway through a chemical substitution reaction. The agonist or activator (such as agonist cGAMP of the STING pathway and derivative thereof) of the innate immune pathway kills tumor cells by activating the STING immune pathway to produce immune T cells, and cisplatin molecules exert anticancer effects by inhibiting DNA replication. The cisplatin metal complex containing an innate immune activator enables the drug molecules to increase effect and decrease toxicity, and exerts two-pronged anti-tumor effects.

The cyclic dinucleotide cGAMP of the present invention refers to 2′3′-cGAMP or Cyclic [G(2′,5′)pA(3′,5′)p] unless otherwise specified.

### DETAILED DESCRIPTION

The present invention is described in detail below through embodiments. In the present invention, the following embodiments are only used for preferably illustrating the present invention, not used for limiting the scope of the present invention.

### Embodiment 1: Preparation of Cyclic Dinucleotide cGAMP and Derivative Thereof

cGAMP or derivative thereof (cGAMPX) is synthesized by cyclic cGMP-AMP dinucleotide synthase (cGAS) through catalysis under the activation conditions after binding DNA according to literature methods. The purity is above 98%. (Li P. W, et al., Immunity, 2013, 39(6), 1019-1031.)

### Embodiment 2: Preparation of Innovative Cisplatin Anti-Tumor Drug

Cisplatin (cis-[PtCl₂(NH₃)₂]), oxaliplatin and related chemical reagents are all purchased from Sigma company. cis-[PtCl₂(NH₃)₂] undergoes a substitution reaction with cGAMP in the DMF solvent to prepare cis-[Pt(cGAMP)(NH₃)₂]; or hydrogen peroxide is added in the chemical reaction for preparation to prepare a tetravalent platinum complex cis-[Pt(cGAMP)(NH₃)₂(Cl)(OH)].

### Embodiment 3: Test of Anti-Tumor Effect of the Novel Cisplatin Anti-Tumor Drug with a Tumor-Bearing Mouse Model, that is, the Inhibition Effect on the Growth of Subcutaneous Transplanted Tumors in Animals

Animals

Genus, Strain, Sex, Weight, Source, Certificate

BALB/c ordinary mice, male, 18-20 g in weight, 6-8 weeks old, SPF grade, purchased from Shanghai SLAC Laboratory Animal Co., Ltd.[Laboratory Animal Quality Certificate Number: SCXK (Shanghai) 2007-0005].

Feeding Conditions

All mice are free to forage and drink, and are fed at room temperature of (23±2)° C. Feed and water are sterilized at high pressure, and all experimental feeding processes are SPF grade.

Dose Setting

Mice are orally administrated, and [cis-Pt(NH₃)₂(cGAMP)] and the tetravalent platinum complex cis-[Pt(cGAMP)(NH₃)₂(Cl)(OH)] are both set to be 6 mg/kg in one dose group.

Test Controls

Negative control: normal saline solution

Positive control: cGAMP, 5 mg/kg in dose (subcutaneous) oxaliplatin, 2 mg/kg in dose (oral)

Administration Method

Administration routes: control group: cGAMP, 5 mg/kg in dose (subcutaneous)

Oxaliplatin, 2 mg/kg in dose (oral);

Immune activator platinum complex, 6 mg/kg in dose (oral)

Administration volume: 100 μl/animal; number of times of administration: continuous administration for 20 days, once every two days.

Number of animals in each group: 10

Tumor Cell Lines

Mouse colorectal cancer cell line CT26, mouse Lewis lung cancer cell line LL/2, human ovarian cancer cell line SK-OV-3, and human melanoma cell line A375, which all purchased from the Cell Bank of the Chinese Academy of Sciences.

Main Test Steps

1. Establishment and Intervention of Mouse Tumor Model

Cells are cultured and passaged, are collected in the logarithmic phase of cells, and are made into cell suspension with a concentration of (1.0×10⁷) per milliliter; 0.2 ml of the cell suspension is injected into the axilla of the right forelimb of each mouse (the number of cells is 2.0×10⁶/mouse), after about 10 days, when growing to about 5 mm, the tumors are successfully induced, and are randomly divided into 5 groups, including: A: negative control group (normal saline group); B, C: positive control group, cGAMP group (subcutaneous administration, 5 mg/kg); oxaliplatin administration group (oral administration, 2 mg/kg); D, E: [cis-Pt(NH₃)₂(cGAMP)] and tetravalent platinum complex cis-[Pt(cGAMP)(NH₃)₂Cl)(OH)] administration group (oral administration, 6 mg/kg). The 5 groups are administered once every two days for 20 consecutive days. 20 days later, the mice are killed and the tumors are weighed. Tumor inhibition rate=[1−mean tumor weight of experimental group/mean tumor weight of group A)]×100%.

2. Statistical Analysis

Data is expressed with x±s, and processed with SPSS10.0 software. The significance of the difference between tumor weights of all groups is compared by a one-way ANOVA test. The significance level a=0.05.

Results

After subcutaneous inoculation of tumor cells in mice, a subcutaneous transplanted tumor model is successfully prepared. The innovative cGAMP-cisplatin anti-tumor drugs [cis-Pt(NH₃)₂(cGAMP)], cis-[Pt(cGAMP)(NH₃)₂(Cl)(OH)], and innate immune pathway activator(cGAMP) or oxaliplatin alone can obviously inhibit the growth of tumors. 20 days after administration, the weights of tumors are all significantly lower than those of the negative control group (P<0.05, P<0.01), and the cGAMP-cisplatin anti-tumor drug is superior to cGAMP or oxaliplatin alone, indicating that the cGAMP-cisplatin anti-tumor drug has a better anti-tumor effect. The specific results are shown in Tables 1-4.

TABLE 1
Effect of cGAMP-cisplatin anti-tumor drug on subcutaneous
transplanted tumor of BalB/C mouse colorectal
cancer cell CT26 (n = 10, mean ± SD)

| | | Mean tumor |
|---|---|---|
| | | inhibition |
| Groups | Mean tumor weight (g) | rate (%) |
| Negative control group | 2.258 ± 0.221 (g) | — |
| cGAMP group | 0.840 ± 0.218 (g)** | 62.8 |
| Oxaliplatin group | 0.963 ± 0.223 (g)** | 57.3 |
| cGAMP-cisplatin group | 0.648 ± 0.216 (g)** | 71.3 |
| cGAMP-tetravalent | 0.689 ± 0.224 (g)** | 69.5 |
| cisplatin group | | |

Note:
*P < 0.05 vs negative control group;
**P < 0.01 vs negative control group.

TABLE 2
Effect of cGAMP-cisplatin anti-tumor drug on subcutaneous
transplanted tumor of C57 mouse Lewis lung cancer
cell line LL-2 n = 10, mean ± SD)

| | | Mean tumor |
|---|---|---|
| | | inhibition |
| Groups | Mean tumor weight (g) | rate (%) |
| Negative control group | 2.560 ± 0.218 (g) | — |
| cGAMP group | 0.980 ± 0.214 (g)** | 56.6 |
| Oxaliplatin group | 0.963 ± 0.223 (g)** | 62.4 |
| cGAMP-cisplatin group | 0.758 ± 0.226 (g)** | 70.4 |
| cGAMP-tetravalent | 0.784 ± 0.232 (g)** | 69.4 |
| cisplatin group | | |

Note:
*P < 0.05 vs negative control group;
**P < 0.01 vs negative control group

TABLE 3
Effect of cGAMP-cisplatin anti-tumor drug on mouse subcutaneous
transplanted tumor of human melanoma cell line A375 (n = 10,
mean ± SD)

| | | Mean tumor |
|---|---|---|
| | | inhibition |
| Groups | Mean tumor weight (g) | rate (%) |
| Negative control group | 2.628 ± 0.186 (g) | — |
| cGAMP group | 0.969 ± 0.216 (g)** | 63.0 |
| Oxaliplatin group | 0.989 ± 0.215 (g)** | 62.4 |
| cGAMP-cisplatin group | 0.786 ± 0.218 (g)** | 70.1 |
| cGAMP-tetravalent | 0.794 ± 0.224 (g)** | 69.8 |
| cisplatin group | | |

Note:
*P < 0.05 vs negative control group;
**P < 0.01 vs negative control group.

TABLE 4
Effect of cGAMP-cisplatin anti-tumor drug on mouse
subcutaneous transplanted tumor of human ovarian
cancer cell line SK-OV-3 (n = 10, mean ± SD)

| | | Mean tumor |
|---|---|---|
| | | inhibition |
| Groups | Mean tumor weight (g) | rate (%) |
| Negative control group | 2.646 ± 0.218 (g) | — |
| cGAMP group | 0.997 ± 0.212 (g)** | 62.3 |
| Oxaliplatin group | 0.989 ± 0.215 (g)** | 62.6 |
| cGAMP-cisplatin group | 0.789 ± 0.218 (g)** | 70.2 |
| cGAMP-tetravalent | 0.813 ± 0.226 (g)** | 69.3 |
| cisplatin group | | |

Note:
*P < 0.05 vs negative control group;
**P < 0.01 vs negative control group.

1. A composition of a novel immune activator platinum anti-tumor compound, comprising: [Pt(NH₃)(cGAMPX)], [Pt(NH₃(cGAMPX)(Cl)(OH)], and homologs or analogs thereof, wherein the cGAMPX refers to an activator or agonist of the innate immune pathway (STING pathway), including but not limited to cyclic dinucleotide (such as 2′3′-cGAMP) and various derivatives thereof, and also including an activator or agonist of the STING passing through high throughput screening and optimization, and various platinum anti-tumor drug molecules prepared from cisplatin drug molecules and a STING pathway immune activator (such as 2′3′-cGAMP or derivative thereof).

2. The novel platinum complex includes but is not limited to immune activator (cGAMPX) cisplatin anti-tumor compounds, as well as includes various platinum compounds formed by a STING immune pathway activator and other platinum complexes, and ruthenium, rhodium, iridium and other metal anti-tumor complexes formed by taking the 2′3′-cGAMP or derivative thereof as a ligand.

3. A preparation method of the immune activator cisplatin anti-tumor drug, comprising: preparing various transition metal complex anti-tumor drug molecules from cisplatin drug molecules (or ruthenium, rhodium, iridium and other metal complexes) and a STING pathway immune activator (such as cGAMP) through a substitution (addition) reaction.

4. An application of the immune activator cisplatin anti-tumor compound in fighting tumors and an application thereof in preparing anti-tumor drugs.

5. An application of the ruthenium (rhodium, iridium) metal complex anti-tumor substance in fighting tumors and an application thereof in preparing anti-tumor drugs.

6. The anti-tumor drug according to claim 1, wherein the anti-tumor drug is applied to treatment of various tumors, including but not limited to colorectal cancer, ovarian cancer, prostate cancer, testicle cancer, lung cancer, nasopharyngeal cancer, esophagus cancer, malignant lymphoma, head and neck cancer, thyroid cancer, osteogenic sarcoma and other various solid tumors.
